(19) United States
(12) Patent Application Publication
(10) Pub. No.: US 2008/0220499 A1
(43) Pub. Date: Sep. 11, 2008

(54) NOVEL PROTEASE FOR INDUSTRIAL APPLICATIONS

(76) Inventors: Smita Shrikant Nilegaonkar, Maharashtra (IN); Vasudeo Pandharinath Zambare, Maharashtra (IN); Pradnya Pralhad Kanekar, Maharashtra (IN); Prashant Kamalakar Dhakephalkar, Maharashtra (IN); Seema Shreepad Sarnaik, Maharashtra (IN); Narasimhan Kannan Chandra Babu, Tamilnadu (IN); Baddipudi Ramaniah, Tamilnadu (IN); Rama Rajaram, Tamilnadu (IN); Thirumalachari Ramasami, Tamilnadu (IN); Yegnisettipalli Krishnaiah Saikumari, Karnatka (IN); Padmanabhan Balaram, Karnatka (IN)

Correspondence Address: LUEDEKA, NEELY & GRAHAM, P.C., P O BOX 1871, KNOXVILLE, TN, 37901, US

(21) Appl. No.: 11/931,537

(22) Filed: Oct. 31, 2007

(30) Foreign Application Priority Data
Nov. 16, 2006 (IN) 2471/DEL/2006

(51) Int. Cl. C12N 9/52

(52) U.S. Cl. 435/220

(57) ABSTRACT

The present invention relates to an extracellular enzyme protease obtained by growing the culture of Pseudomonas aeruginosa MCM B-327 isolated from vermiculture pit soil and deposited in MTCC, IMTECH, Chandigarh with designation MTCC 5270, in production medium of pH 7.0; containing soybean meal and tryptone as raw materials, at 30° C. for 72 h. The organism was also able to produce protease using different agricultural products/byproducts as protein sources. The partially purified non-collagenolytic, calcium independent protease with molecular weight 60 kDa has activity in pH range of 6.0-11.0 and temperature range of 25-65° C.; stability in pH range of 6.0-10.0 and temperature 25-45° C. The protease activity was retained for 8 months when stored at ambient temperature. Ammonium sulphate precipitated enzyme was able to completely dehair animal skins and hides without chemicals like lime, sodium sulphide and calcium.

### FIELD OF INVENTION

The present invention relates to a novel protease for industrial applications. More particularly, the present invention relates to a novel protease produced from a bacterial strain of Pseudomonas aeruginosa MCM B-327 deposited at MTCC, IMTECH, Chandigarh, India and designated as MTCC 5270.

### BACKGROUND OF INVENTION

Proteases are the most important enzymes from industrial point of view. The protease of the present invention finds enormous potential application in leather processing industry as an eco-benign option for dehairing of hides and skins without using any chemicals. It may also be used in soaking process of leather manufacturing. Further, the enzyme finds application as an additive in detergent and also for recovery of silver from waste X-ray and photographic films. It may also be used for preparations of protein hydrolysates. Yet another potential application of the protease of the present invention is in pharmaceutical industries and food processing industry.

### DESCRIPTION OF THE PRIOR ART

Proteases are conventionally produced from several micro-organisms, plant sources and animal sources. However, commercial production of protease from bacterial and fungal origin have gained much importance because of their extracellular production, submerged cultivation, high yield and short duration of production and easy recovery of the enzyme.

Several bacteria produce protease and the major producers belong to genus Bacillus. Many fungi belonging to genera Aspergillus, Cephalosporium, Fusarium, Paecilomyces, Penicillium etc. and species like Chrysosporium keratinophilum, Conidiobolus coronatus, Entomophthora coronata, Rhizopus oryzae, Scedosporium apiospermum, Tritirachium album etc. produce protease, as reported by Ganesh Kumar and Takagi (Biotechnology Advance 17, 561-594, 1999). Chitte et al., (Letters in Applied Microbiology 28, 131-136, 1999); Yang and Wang (Botanical Bulletin of Academia Sinica 40, 259-265, 1999); Bressollier et al., (Applied and Environmental Microbiology 65 (6), 2570-2576, 1999) have reported the production of protease from Streptomyces species and their biotechnological applications.

There have been several reports on the different features of the proteases available in the public domain. They are summarized below.

### Optimum pH and Temperature

Many proteases have high thermal and broad pH stability particularly in alkaline pH along with stability of the enzyme in bleaching agents, solvents, detergents and surfactants, as reported by Joo and Chang (Process Biochemistry 40, 1263-1270, 2005), Ramani et al., (Process Biochemistry 40, 3352-3359, 2005), Chauhan and Gupta (Process Biochemistry 39, 2115-2122, 2004), Joo et al., (Process Biochemistry 39, 1441-1447, 2004).

The optimum pH range of alkaline proteases is generally between pH 9 and 11 as reported by Kanekar et al., (Bioresource Technology 85, 87-93, 2002); Nilegaonkar et al., (World Journal of Microbiology and Biotechnology 18, 785-789, 2002); Kanekar et al., (Indian Patent 188072); with a few exceptions of higher pH optima of 11.5, Yum et al., (Bioscience Biotechnology and Biochemistry 58, 470-474, 1994); Takami et al., (Applied Microbiology and Biotechnology 33, 519-523, 1990), pH 11-12, as reported by Horikoshi, (Agricultural and Biological Chemistry 35, 1407-1414, 1971); pH 12.3, as reported by Kobayashi et al., (Applied Microbiology and Biotechnology 43, 473-481, 1995).

The optimum temperature of alkaline proteases ranges from 50 to 70° C. However, the enzyme from an alkalophilic Bacillus sp. B189 shows an exceptionally high optimum temperature of 85° C. Alkaline proteases from Bacillus sp., Streptomyces sp. and Thermus sp. are quite stable at high temperatures, and the addition of CaCl₂ further enhances enzyme thermostability.

### Molecular Masses

The molecular masses of alkaline proteases range from 15 to 30 kDa, as reported by Gupta et al., (Applied Microbiology and Biotechnology 59, 15-32, 2002) with few reports of higher molecular masses of 31.6 kDa; as reported by Freeman et al., (Biochemistry Journal 295, 463-469, 1993), 33 kDa as reported by Larcher et al., (Biochemistry Journal 315, 119-126, 1996); 36 kDa, as reported by Tsujibo et al., Journal of Applied Bacteriology 69, 520-529, 1990) and 45 kDa, as reported by Kwon et al., (Biotechnology Letters 16, 413-418, 1994). However, an enzyme from Kurthia spiroforme had an extremely low molecular weight of 8 kDa, as reported by Steele et al., (Enzyme and Microbial Technology 14, 358-360, 1992). Kobayashi et al., (Applied Microbiology and Biotechnology 45, 63-71, 1996).

### Metal Ion Requirement and Inhibitors

Alkaline proteases require a divalent cation like Ca⁺², Mg⁺², and Mn⁺² or a combination of these cations for maximum activity. It is believed that these cations protect the enzyme against thermal denaturation and play a vital role in maintaining the active conformation of the enzyme at high temperatures, as reported by Steele et al., (Enzyme and Microbial Technology 14, 358-360, 1992). In addition, specific Ca⁺² binding sites that influence the protein activity and stability apart from the catalytic site are described for proteinase, as reported by Bajorath et al., (European Journal of Biochemistry 176, 441-447, 1988). In some of the studies, catalytic activity was inhibited by Hg ions, as reported by Rahman et al., (Applied Microbiology and Biotechnology 40, 822-827, 1994).

### Substrate Specificity

Although alkaline proteases are active against many synthetic substrates as well as native proteins, reaction rates vary widely. The alkaline proteases and/or subtilisins are found to be more active against casein than against hemoglobin or bovine serum albumin.

Ogino et al., (Journal of Bioscience and Bioengineering 87 (1), 61-68, 1999) observed that the protease from P. aeruginosa PST-01 hydrolyzed elastin and elastase-specific substrates like succinyl-Ala-Ala-Ala-p-nitroanilide, succinyl-Ala-Pro-Ala-p-nitroanilide, succinyl-Ala-Ala-Pro-Leu-p-nitroanilide and glutaryl-Ala-Ala-Pro-Leu-p-nitroanilide at a faster rate. The enzymatic cleavage of keratin can be accompanied by a simultaneous reduction of disulfide bonds. A thermostable alkaline protease from an alkalophilic Bacillus sp. no. AH-101 exhibiting keratinolytic activity showed degradation of human hair keratin with 1% thioglycolic acid at pH 12 and 70° C. Similarly, enhanced keratin degradation after addition of DTT has also been reported by Böckle et al., (Applied and Environmental Microbiology 61, 3705-3710, 1995) and for alkaline proteases of Streptomyces sp.

Stability of the protease against different organic solvents (such as methanol, 1-propanol, 2-propanol, ethylene glycol, ethyl acetate, acetone, xylene, toluene, benzene) in reaction mixture of each solvent has been studied by Najafi et al., (Electronic Journal of Biotechnology 8 (2), 197-203, 2005) at 55° C. for 20 min using casein as substrate. The enzyme showed 80-90% stability for 20% of methanol, ethylene glycol, xylene and toluene. Stability and activity of the enzyme in organic solvents signifies its use in peptide bond synthesis as reported by Ogino et al., (Journal of Bioscience and Bioengineering 88 (5), 513-518, 1999).

Protease has application in leather industry because of its broad pH activity and stability (pH 6-10), substrate specificity to casein, elastin, albumin, globuline but not to keratin and collagen. The removal of epidermis layer is due to the hydrolysis of the cementing substance present in between the epidermis and dermis layers of skin/hide. A novel keratinase from Bacillus subtilis with potential dehairing activity on goat skin was reported by Alexandre et al., (Applied and Environmental Microbiology 71, 594-596, 2005). It completed the dehairing in 9 hr at pH 9.0, 24° C., with 4.8 U g⁻¹ of skin without sodium sulphide. It has high keratinase but no colagenase activity. Nilegaonkar et al., (Bioresource Technology 2006, in press) reported a non-collagenase type protease from Bacillus cereus MCM B-326 for the dehairing of buffalo hide in tap water with 1% enzyme at room temperature (28±2° C.). A protease obtained from Conidiobollus coronatus has potential in soaking, dehairing and bating of animal skins/hide as reported by Laxman et al., (U.S. Pat. No. 6,777,219). A mixture of proteolytic enzymes derived from Streptomyces griseus, was used to unhair bovine hide pieces by 30-min pretreatment of the pieces with carbonate buffer, as reported by Gehring et al., (Journal of American Leather Chemists Association 97, 406-411, 2002). Yeshodha et al., (Leather Science, 25, 36-45, 1978) developed a process for the manufacture of grain garment leather using Jawasee plant protease obtained from leaves and bark of Jawasee shrub.

As reported by Bayoudh et al., (Journal of Industrial Microbiology and Biotechnology 24, 291-295, 2000) protease from Pseudomonas aeruginosa has industrial application in detergents because of broad pH and temperature activity as well as stability. Wang and Chio (Enzyme and Microbial Technology 22, 629-633, 1998) reported the proteolytic enzymes from P. aeruginosa K-187 to deproteinze the shrimps and shell wastes. Reference may also be made to Najafi et al., (Electronic Journal of Biotechnology 8 (2), 197-203, 2005), who produced protease from Pseudomonas aeruginosa. The resulting protease was able to dehair cow hide within 2.30-3 h at 50° C. but after further incubation it digested the hide collagen due to high collagenolytic activity which is not desirable in leather industry.

The major limitations associated with the enzymatic processes relate to the following parameters, which have to be matched with the industrial requirements.
a) Substrate specificity: e.g. the enzyme from Pseudomonas aeruginosa reported in prior art by Najafi et al., (Electronic Journal of Biotechnology 8 (2), 197-203, 2005) is collagenolytic and hence not suitable in leather industry.
b) Economy of application: e.g. the enzyme from Pseudomonas aeruginosa reported in prior art by Najafi et al., (Electronic Journal of Biotechnology 8 (2), 197-203, 2005) requires higher temperature (50° C.) for dehairing of hide hence not economical.

The proteases from other organisms reported in prior art require buffers for application in leather industry. Some also require chemical assistance. Many of the reported enzymes have specific applications and may be useful for only certain industries.

Thus, a protease having broad application range and accordingly useful in many industrial activities is still the need of the day.

### OBJECTIVES OF THE PRESENT INVENTION

The main object of the present invention is therefore to provide a novel protease for industrial applications, which obviates the limitations as stated above.

Another object of the present invention is to provide a protease that does not exhibit collagenolytic activity.

Yet another object of the present invention is to provide a protease which is active at broad pH and temperature range and has storage stability at ambient temperature.

Still another objective of the present invention is to provide a protease which is produced with cheap substrates so as to reduce the cost of production.

Another object of the present invention is to provide a protease that is stable in presence of SDS, sodium tripolyphosphate, sodium tetraborate, tween 80, triton X100 which are ingredients of commercial detergents and surfactants. It is also active in proprietary commercial detergents like Tide®, Ariel®, Nirma®, Rin Shakti® and Surf Excel®.

Yet another object of the present invention is to provide a protease which is calcium ion independent for its activity and/or stability.

Still another object of the present invention is to provide a protease useful in dehairing of animal skins and hides, as a detergent additive, in preparation of protein hydrolysate and recovery of silver from X-Ray and photographic films.

A further object of the present invention is to provide a protease that improves the leather quality, reduce the pollution load of the tannery effluent and avoid the health hazards to the tannery workers.

Still another object of the present invention is to provide a protease that results in dehairing of animal hide from hair root yielding intact hair that can be used as a byproduct.

Yet another object of the present invention is to provide a protease that hydrolyzes albumin hence could be used in soaking of an animal hide.

Still another object of the present invention is to provide a process to prepare a protease from a bacterial strain, Pseudomonas aeruginosa MCM B-327, isolated from vermicompost pit soil, Pune, Maharashtra, India and deposited in MTCC, IMTECH, Chandigarh with a designation number MTCC 5270.

### SUMMARY OF THE INVENTION

Accordingly, the present invention provides a novel protease from a bacterial strain of Pseudomonas aeruginosa MCM B-327, deposited at MTCC, IMTECH, Chandigarh, India and designated as MTCC 5270, having molecular weight in the range of 55 to 70 kDa; wherein the said protease exhibits specificity against globular type of proteins; the said protease having stability in pH range of 6.0 to 10.0 and temperature ranging from 25 to 45 degree C.; activity in pH range of 6.0 to 11.0 and temperature ranging from 25 to 65 degree C.; wherein the said protease is calcium ion independent for its activity and/or stability; the said protease exhibits inhibition by metals Cu⁺², Zn⁺², Hg⁺¹, and solvents; and the said protease further exhibits inertness to collagen.

The present invention further provides a process for the preparation of a novel protease, wherein the process steps comprises:
a) growing Pseudomonas aeruginosa MCM B-327, deposited at MTCC, IMTECH, Chandigarh and designated as MTCC 5270 in a production medium containing essentially a protein source, an organic nitrogenous source, maintained at a pH in the range of 6.0 to 9.0, in submerged culture condition, at temperature ranging from 25 to 40 degree C., under shaking condition to obtain a culture broth;
b) harvesting the culture broth obtained from step[a] after a period in the range of 48 to 72 hrs followed by separation of the enzyme in liquid phase by salting out method to obtain the novel protease

In an embodiment of the present invention, the bacterial strain used is isolated from vermicompost pit soil, Pune, Maharashtra, India.

In another embodiment of the present invention, the protein source used for the production medium may be selected from agricultural products/byproducts such as deoiled ground nut cake, saffola cake, rape seed cake, soybean cake, soya flour, soybean meal, bengal gram flour, wheat bran, either individually or in any combination.

In still another embodiment of the present invention, the organic nitrogenous source used for the culture medium may be selected from beef extract, yeast extract, peptone, tryptone and casein either individually or in any combination.

In yet another embodiment of the present invention, the method of separation used may be such as ammonium sulphate precipitation.

In still another embodiment of the present invention, the types of proteins exhibiting specificity against the protease may be such as casein, bovine serum albumin (BSA), gelatin, azocasein, azoalbumin, azocoll.

In yet another embodiment of the present invention, the stability of the protease is independent on the divalent metal ions such as Ca⁺², Mg⁺².

In still another embodiment of the present invention, the metal showing inhibition in respect of the protease may be such as Cu⁺², Zn⁺², Hg⁺¹.

In yet another embodiment of the present invention, the solvents showing inhibition in respect of the protease may be such as methanol, benzene, 1-pentanone and ethanol.

In yet another embodiment of the present invention, the fibrous protein-collagen towards which the protease shows inertness.

### BRIEF DESCRIPTION OF THE DRAWINGS

Further advantages of exemplary embodiments disclosed herein may become apparent by reference to the detailed description of preferred embodiments when considered in conjunction with the drawings, wherein:

FIGS. 1-2 are graphs of effects of pH on enzyme activity according to the disclosure; and

FIGS. 3-4 are graphs of effects of temperature on enzyme activity according to the disclosure.

### DETAILED DESCRIPTION OF THE INVENTION

The production medium for the preparation of the protease is prepared using essentially a protein source and organic nitrogenous source. The medium is maintained at a pH in the range of 6.0 to 9.0. The bacterial strain of Pseudomonas aeruginosa MCM B-327, deposited at MTCC, IMTECH, Chandigarh and having designation no. MTCC 5270 is grown in the said production medium for a period of 48-72 hrs in submerged culture condition with shaking. The medium is harvested by centrifugation at 13,000×g. The protease activity of the supernatant was determined by caseinolytic assay as follows. The cell free supernatant (1 ml) was mixed with 4 ml of casein (0.625% w/v) and incubated at 37° C. for 30 min. The reaction was stopped by addition of 5 ml of 5% trichloroacetic acid. Enzymatically hydrolyzed casein was measured by modified Folin Ciocalteu method against casein treated with inactive enzyme as blank. A standard graph was generated using standard tyrosine solutions of 5-50 μg ml⁻¹. One unit of protease activity was defined as the amount of enzyme which liberated 1 μg tyrosine per min at 37° C. The enzyme was partially purified by 80% saturation of ammonium sulphate precipitation method to obtain the novel protease.

The casein hydrolyzing activity of the enzyme is defined in terms of tyrosine equivalents. The partially purified enzyme is stable in pH range of 6-11 and temperature range of 25-65° C. At ambient temperature (28±2° C.) when ammonium sulphate precipitate was stored 8 months, 100% activity was retained.

The inventiveness of the present invention lies in providing a protease of molecular weight as high as 60 kDa having no collagenolytic activity.

The following provides a comparative data relating to the protease of the present invention and that produced by Najafi et al (Electronic Journal of Biotechnology 8, 2005) from a different strain of Pseudomonas aeruginosa.

Comparative Account of Protease from P. aeruginosa with Respect to its Production, Properties and Application

| | | | Enzyme relating to P. aeruginosa |
|---|---|---|---|
| Sr. | | Enzyme relating to P. aeruginosa | MTCC 5270 |
| No. | Properties | PD100 of the prior art | of the present invention |
| 1 | Production medium | CYKN (casein, yeast extract, | ST (soybean meal, tryptone), |
| | | K₂HPO₄, NaCl) | & Gram flour-soya flour |
| 2 | Production pH | 7.5 | 6.0-9.0, optimum 7.0 |
| 3 | Production | Not mentioned | 25-40° C., optimum 30° C. |
| | temperature | | |
| 4 | Production time | 24 hrs | 48 to 72 hrs |
| 5 | Optimum pH for | 8.0 | 8.0 |
| | activity | | |
| 6 | pH stability | 6.5-11 | 6-11 |
| 7 | Optimum temperature | 60° C. | 37° C. |
| | for activity | | |
| 8 | Temperature stability | 55° C. | 25-65° C. |
| 9 | Substrate specificity | collagen, fibrin, azocasein, casein, | casein, azocasein, azocoll, |
| | | hemoglobin, BSA, ovalbumin, | azoalbumin, BSA, gelatin. |
| | | elastin | |
| 10 | Inhibitors | Ag⁺², Ni⁺², Cu⁺², Zn⁺², β-ME, | Hg⁺¹, Cu⁺², Zn⁺², DTT, H₂O₂ |
| | | DTNB,, DEPC, Iodoacetamide, | |
| | | HNBB, NEM | |
| 11 | Active site | Enzyme has thiol group at active | Enzyme has no thiol or |
| | | site | serine group at active site |
| 12 | Molecular weight | 38-36 kDa | ~ 60 kDa |
| 13 | Application | Dehairing of cow hide | Dehairing of buffalo hide, |
| | | | cow hide, goat skin, sheep |
| | | | skin |
| 14 | Application pH | Alkaline (pH 8.0) | Neutral (pH 7.0) |
| 15 | Application | 50° C. | Ambient temperature (10-40° C.) |
| | temperature | | |
| 16 | Application | Tris-HCl Buffer | Water |
| | solvent/buffer | | |
| 17 | Application time | 2.30-3 hrs | 5-21 hrs |
| 18 | Side effect | Degradation of skin, due to | NIL since the isolated |
| | | collagenolytic activhy | protease has no |
| | | | collagenolytic activity |
| 19 | Other applications | Waste treatment, detergent | Detergent, Recovery of Ag |
| | | | from waste photo film, |
| | | | preparation of protein |
| | | | hydrolysate |

The following examples are given by way of illustration only and therefore should not be construed to limit the scope of the present invention.

### EXAMPLE-1

This example illustrates the preparation of the protease using soybean meal+tryptone (HiMedia) at flask level. Cells of Pseudomonas aeruginosa MCM B-327 were transferred from glycerol stock (1 vial of 1 ml) into 10 ml nutrient broth (peptone, beef extract, yeast extract, sodium chloride) and incubated 21-24 h at 30° C. under shaking culture condition. One ml of the cell broth was then inoculated to 150 ml nutrient agar and incubated at 30° C. for 21-24 h. The cell growth was suspended in 15 ml sterile saline and 1 ml (10⁹ cells ml⁻¹) was inoculated in 100 ml production medium prepared by incorporating 1 g soybean meal+1 g tryptone in 100 ml distilled water and having pH 7.0. The medium was incubated at 30° C. for 72 hrs under shaking condition (150 rpm). The protease activity was determined by caseinolytic assay method. The activity of the cell free supernatant was found to be 324 U ml⁻¹ min⁻¹ at 72 hrs.

### EXAMPLE-2

This example illustrates the preparation of the protease using marketed soybean flour at flask level. Cells of Pseudomonas aeruginosa MCM B-327 were transferred from glycerol stock (1 vial of 1 ml) into 10 ml nutrient broth (peptone, beef extract, yeast extract, sodium chloride) and incubated 21-24 h at 30° C. under shaking culture condition. One ml of the cell broth was then inoculated to 150 ml nutrient agar and incubated at 30° C. for 21-24 h. The cell growth was suspended in 15 ml sterile saline and 1 ml (10⁹ cells ml⁻¹) was inoculated in 100 ml production medium prepared by incorporating 1 g marketed soybean flour in 100 ml distilled water and having pH 7.0. The medium was incubated at 30° C. for 72 hrs under shaking condition at 150 rpm. The protease activity was determined by caseinolytic assay method. The activity of the cell free supernatant was 296 U ml⁻¹ min⁻¹ at 72 hrs.

### EXAMPLE-3

This example illustrates the preparation of the protease using bengal gram flour at flask level. Cells of Pseudomonas aeruginosa MCM B-327 were transferred from glycerol stock (1 vial of 1 ml) into 10 ml nutrient broth (peptone, beef extract, yeast extract, sodium chloride) and incubated 21-24 h at 30° C. under shaking culture condition. One ml of the cell broth was then inoculated to 150 ml nutrient agar and incubated at 30° C. for 21-24 h. The cell growth was suspended in 15 ml sterile saline and 1 ml (10⁹ cells ml⁻¹) was inoculated in 100 ml production medium prepared by incorporating 1 g bengal gram flour in 100 ml distilled water and having pH 7.0. The medium was incubated at 30° C. for 72 hrs under shaking condition (150 rpm). The protease activity was determined by caseinolytic assay method. The activity of the cell free supernatant was 181 U ml⁻¹ min⁻¹ at 72 hrs.

### EXAMPLE-4

This example illustrates the preparation of the protease using bengal gram flour+wheat bran at flask level. Cells of Pseudomonas aeruginosa MCM B-327 were transferred from glycerol stock (1 vial of 1 ml) into 10 ml nutrient broth (peptone, beef extract, yeast extract, sodium chloride) and incubated 21-24 h at 30° C. under shaking culture condition. One ml of the cell broth was then inoculated to 150 ml nutrient agar and incubated at 30° C. for 21-24 h. The cell growth was suspended in 15 ml sterile saline and 1 ml (10⁹ cells ml⁻¹) was inoculated in 100 ml production medium prepared by incorporating 1 g bengal gram flour+1 g wheat bran in 100 ml distilled water and having pH 7.0. The medium was incubated at 30° C. for 72 hrs under shaking condition (150 rpm). The protease activity was determined by caseinolytic assay method. The activity of the cell free supernatant was 196 U ml⁻¹ min⁻¹ at 72 hrs.

### EXAMPLE-5

This example illustrates the preparation of the protease using bengal gram flour+tryptone at flask level. Cells of Pseudomonas aeruginosa MCM B-327 were transferred from glycerol stock (1 vial of 1 ml) into 10 ml nutrient broth (peptone, beef extract, yeast extract, sodium chloride) and incubated 21-24 h at 30° C. under shaking culture condition. One ml of the cell broth was then inoculated to 150 ml nutrient agar and incubated at 30° C. for 21-24 h. The cell growth was suspended in 15 ml sterile saline and 1 ml (10⁹ cells ml⁻¹) was inoculated in 100 ml production medium prepared by incorporating 1 g bengal gram flour+1 g tryptone in 100 ml distilled water and having pH 7.0. The medium was incubated at 30° C. for 72 hrs under shaking condition (150 rpm). The protease activity was determined by caseinolytic assay method. The activity of the cell free supernatant was 309U ml⁻¹ min⁻¹ at 72 hrs.

### EXAMPLE-6

This example illustrates the preparation of the protease using bengal gram flour+defatted soybean cake at flask level. Cells of Pseudomonas aeruginosa MCM B-327 were transferred from glycerol stock (1 vial of 1 ml) into 10 ml nutrient broth (peptone, beef extract, yeast extract, sodium chloride) and incubated 21-24 h at 30° C. under shaking culture condition. One ml of the cell broth was then inoculated to 150 ml nutrient agar and incubated at 30° C. for 21-24 h. The cell growth was suspended in 15 ml sterile saline and 1 ml (10⁹ cells ml⁻¹) was inoculated in 100 ml production medium prepared by incorporating 1 g bengal gram flour+1 g defatted soybean in 100 ml distilled water and having pH 7.0. The medium was incubated at 30° C. for 72 hrs under shaking condition (150 rpm). The protease activity was determined by caseinolytic assay method. The activity of the cell free supernatant was 341 U ml⁻¹ min⁻¹ at 72 hrs.

### EXAMPLE-7

This example illustrates the preparation of the protease using wheat bran+tryptone at flask level. Cells of Pseudomonas aeruginosa MCM B-327 were transferred from glycerol stock (1 vial of 1 ml) into 10 ml nutrient broth (peptone, beef extract, yeast extract, sodium chloride) and incubated 21-24 h at 30° C. under shaking culture condition. The cell broth was then transferred to nutrient agar and incubated at 30° C. for 21-24 h. One ml of the cell broth was then inoculated to 150 ml nutrient agar and incubated under same conditions. The cell growth was suspended in 15 ml sterile saline and 1 ml (10⁹ cells ml⁻¹) was inoculated in 100 ml production medium prepared by incorporating 1 g wheat bran+1 g tryptone in 100 ml distilled water and having pH 7.0. The medium was incubated at 30° C. for 72 hrs under shaking condition (150 rpm). The protease activity was determined by caseinolytic assay method. The activity of the cell free supernatant was 385 U ml⁻¹ min⁻¹ at 72 hrs.

### EXAMPLE-8

This example illustrates the preparation of the protease using beef extract at flask level. Cells of Pseudomonas aeruginosa MCM B-327 were transferred from glycerol stock (1 vial of 1 ml) into 10 ml nutrient broth (peptone, beef extract, yeast extract, sodium chloride) and incubated 21-24 h at 30° C. under shaking culture condition. One ml of the cell broth was then inoculated to 150 ml nutrient agar and incubated at 30° C. for 21-24 h. The cell growth was suspended in 15 ml sterile saline and 1 ml (10⁹ cells ml⁻¹) was inoculated in 100 ml production medium prepared by incorporating 1 g beef extract in 100 ml distilled water and having pH 7.0. The medium was incubated at 30° C. for 72 hrs under shaking condition (150 rpm). The protease activity was determined by caseinolytic assay method. The activity of the cell free supernatant was 196 U ml⁻¹ min⁻¹ at 72 hrs.

### EXAMPLE-9

This example illustrates the preparation of the protease using yeast extract at flask level. Cells of Pseudomonas aeruginosa MCM B-327 were transferred from glycerol stock (1 vial of 1 ml) into 10 ml nutrient broth (peptone, beef extract, yeast extract, sodium chloride) and incubated 21-24 h at 30° C. under shaking culture condition. One ml of the cell broth was then inoculated to 150 ml nutrient agar and incubated at 30° C. for 21-24 h. The cell growth was suspended in 15 ml sterile saline and 1 ml (10⁹ cells ml⁻¹) was inoculated in 100 ml production medium prepared by incorporating 1 g yeast extract in 100 ml distilled water and having pH 7.0. The medium was incubated at 30° C. for 72 hrs under shaking condition (150 rpm). The protease activity was determined by caseinolytic assay method. The activity of the cell free supernatant was 127 U ml⁻¹ min⁻¹ at 72 hrs.

### EXAMPLE-10

This example illustrates the preparation of the protease using peptone at flask level. Cells of Pseudomonas aeruginosa MCM B-327 were transferred from glycerol stock (1 vial of 1 ml) into 10 ml nutrient broth (peptone, beef extract, yeast extract, sodium chloride) and incubated 21-24 h at 30° C. under shaking culture condition. One ml of the cell broth was then inoculated to 150 ml nutrient agar and incubated at 30° C. for 21-24 h. The cell growth was suspended in 15 ml sterile saline and 1 ml (10⁹ cells ml⁻¹) was inoculated in 100 ml production medium prepared by incorporating 1 g peptone in 100 ml distilled water and having pH 7.0. The medium was incubated at 30° C. for 72 hrs under shaking condition (150 rpm). The protease activity was determined by caseinolytic assay method. The activity of the cell free supernatant was 211 U ml⁻¹ min⁻¹ at 72 hrs.

### EXAMPLE-11

This example illustrates the preparation of the protease using tryptone at flask level. Cells of Pseudomonas aeruginosa MCM B-327 were transferred from glycerol stock (1 vial of 1 ml) into 10 ml nutrient broth (peptone, beef extract, yeast extract, sodium chloride) and incubated 21-24 h at 30° C. under shaking culture condition. One ml of the cell broth was then inoculated to 150 ml nutrient agar and incubated at 30° C. for 21-24 h. The cell growth was suspended in 15 ml sterile saline and 1 ml (10⁹ cells ml⁻¹) was inoculated in 100 ml production medium prepared by incorporating 1 g tryptone in 100 ml distilled water and having pH 7.0. The medium was incubated at 30° C. for 72 hrs under shaking condition (150 rpm). The protease activity was determined by caseinolytic assay method. The activity of the cell free supernatant was 100 U ml⁻¹ min⁻¹ at 72 hrs.

### EXAMPLE-12

This example illustrates the substrate specificity towards casein, of the protease isolated from Pseudomonas aeruginosa MCM B-327 when grown in soybean meal−tryptone (HiMedia) medium. The cell free supernatant was used for ammonium sulphate precipitation (80% saturation) of protease. The enzyme was dialyzed against 50 mM Tris-HCl buffer of pH 8.0. The dialyzed enzyme hydrolyzed casein, with activity 2427 Uml⁻¹ min⁻¹.

### EXAMPLE-13

This example illustrates the substrate specificity towards Bovine Serum Albumin (BSA), of the protease isolated from Pseudomonas aeruginosa MCM B-327 when grown in soybean meal−tryptone (HiMedia) medium. The cell free supernatant was used for precipitation of protease. The enzyme was dialyzed against 50 mM Tris-HCl buffer of pH 8.0. The dialyzed enzyme hydrolyzed Bovine serum albumin (BSA), with activity 773 Uml⁻¹ min⁻¹.

### EXAMPLE-14

This example illustrates the substrate specificity towards azocasein, of the protease isolated from Pseudomonas aeruginosa MCM B-327 when grown in soybean meal−tryptone (HiMedia) medium. The cell free supernatant was used for precipitation of protease. The enzyme was dialyzed against 50 mM Tris-HCl buffer of pH 8.0. The dialyzed enzyme hydrolyzed azocasein, with activity 121 Uml⁻¹ min⁻¹.

### EXAMPLE-15

This example illustrates the substrate specificity towards azocoll, of the protease isolated from Pseudomonas aeruginosa MCM B-327 when grown in soybean meal−tryptone (HiMedia) medium. The cell free supernatant was used for precipitation of protease. The enzyme was dialyzed against 50 mM Tris-HCl buffer of pH 8.0. The dialyzed enzyme hydrolyzed azocoll, with activity 384 Uml⁻¹ min⁻¹.

### EXAMPLE-16

This example illustrates the substrate specificity towards azoalbumin, of the protease isolated from Pseudomonas aeruginosa MCM B-327 when grown in soybean meal−tryptone (HiMedia) medium. The cell free supernatant was used for precipitation of protease. The enzyme was dialyzed against 50 mM Tris-HCl buffer of pH 8.0. The dialyzed enzyme hydrolyzed azoalbumin, with activity 77 Uml⁻¹ min⁻¹.

### EXAMPLE-17

This example illustrates the substrate specificity towards gelatin, of the protease isolated from Pseudomonas aeruginosa MCM B-327 when grown in soybean meal−tryptone (HiMedia) medium. The cell free supernatant was used for precipitation of protease. The enzyme was dialyzed against 50 mM Tris-HCl buffer of pH 8.0. The dialyzed enzyme hydrolyzed gelatin, with activity 698 Uml⁻¹ min⁻¹.

### EXAMPLE-18

This example illustrates the mass spectral analysis using MALDI-MS of Collagenase substrate I for detection of collagenase activity in crude enzyme of P. aeruginosa MCM B-327.

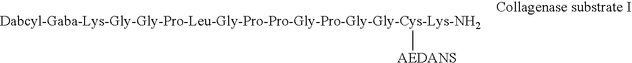

Fluorescence experiment was carried out in triethanolamine buffer (TEA), pH 8 at room temperature. Typically, the assay contained 294 μl of TEA buffer 100 mM pH 8, having substrate concentration of 1.6 μM (5 ul of 80 mM stock solution). The reaction was initiated by adding 5 μl of crude enzyme after 10-fold dilution. The excitation wavelength was 340 nm and the fluorescence intensity was measured at the emission wavelength of 490 nm at different time intervals.

The crude enzyme was screened for true collagenase activity against Collagenase substrate I and found to be inactive, showing non-collagenase nature of the enzyme.

### EXAMPLE-19

This example illustrates the production of protease using 10 L glass fermentor. Cells of Pseudomonas aeruginosa MCM B-327 from glycerol stock (1 vial of 1 ml) were transferred into 10 ml nutrient broth (peptone, beef extract, yeast extract, sodium chloride) and incubated at 30° C. under shaking culture condition. After 21-24 hrs, culture broth was transferred to 700 ml nutrient broth and incubated under same conditions. The culture broth (700 ml) was transferred to 7 L production medium containing (g/L) soybean meal−10 and tryptone-10. The fermentor was run at 30° C. for 48 hrs with agitation of 250 rpm and aeration of 0.75 vvm. The activity in the cell free supernatant was 769 Uml⁻¹ min⁻¹ at 48 hrs. The specific activity of ammonium sulphate precipitated enzyme was 1017 U mg⁻¹ protein.

### EXAMPLE-20

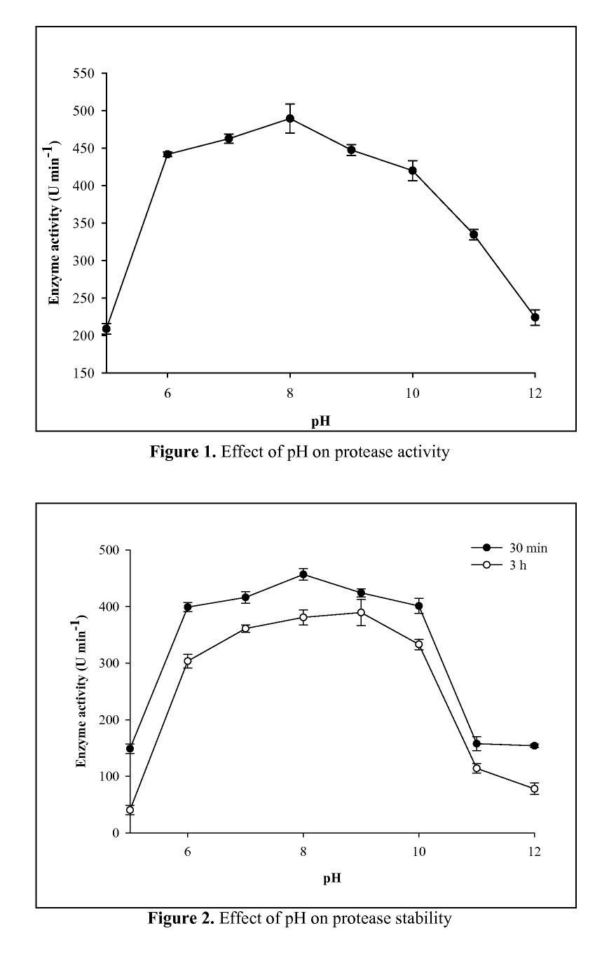

This example illustrates the effect of pH on activity and stability of the protease isolated from Pseudomonas aeruginosa MCM B-327 when grown in soybean meal−tryptone (HiMedia) medium. The protease from cell free supernatant was salt precipitated and its activity as well as stability at different pH was studied. The optimum pH for protease activity was 8.0, determined at 37° C. Activity declined after pH 8.0 and was 85% and 70% of the maximum at pH 10.0 and 11.0, respectively. The enzyme was active even at pH 12 (FIG. 1).

The pH stability of the protease was determined by incubating the enzyme for 30 min and 3 hr at pH range of 5 to 12. The data presented in FIG. 2 show that the enzyme was stable in pH range of 6-10. The enzyme retained 85% and 70% of its activity at 37° C., for pH 9.0 and 10.0, respectively up to 3 h.

### EXAMPLE-21

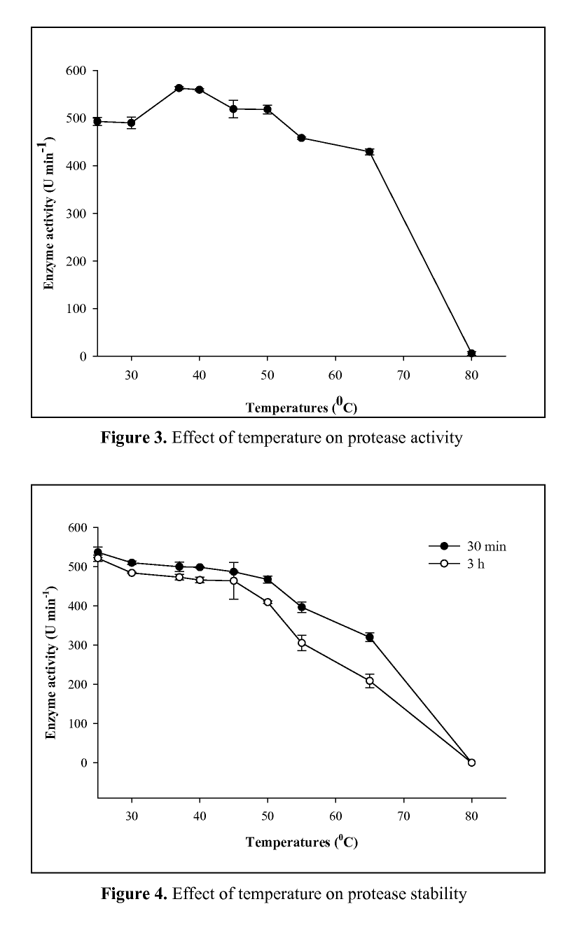

This example illustrates the effect of temperature on activity and stability of the protease isolated from Pseudomonas aeruginosa MCM B-327 when grown in soybean meal−tryptone (HiMedia) medium. The cell free supernatant was salt precipitated and used for temperature activity as well as stability. The optimum temperature for protease activity was determined by varying the reaction temperature at pH 8.0. Enzyme activity was estimated between 25 and 80° C. The temperature optimum of the proteolytic activity was 37° C. The enzyme had 90% and 75% of the maximum activity at 50° C. and 65° C., respectively (FIG. 3) with sharp decrease in activity after 65° C.

The thermal stability of the protease was determined by incubating the enzyme for 30 min and 3 hr at different temperatures in 50 mM Tris-HCl (pH 8.0). The data presented in FIG. 4 show that the enzyme was stable up to 45° C. followed by a rapid loss of activity after 55° C. The enzyme retained more than 75% and 50% activity at 55° C. for 30 min and 3 h respectively. However, the enzyme was completely inactivated at 80° C.

### EXAMPLE-22

This example illustrates the effect of metal ions on activity of the protease isolated from Pseudomonas aeruginosa MCM B-327 when grown in soybean meal−tryptone (HiMedia) medium. The cell free supernatant was salt precipitated and used for this study in Tris-HCl buffer, pH 8.0 at 37° C. The enzyme was resistant to inhibition by Ca⁺⁺, Fe⁺⁺, Na⁺, Mg⁺⁺ and Mn⁺⁺; however, Cu⁺⁺, Zn⁺⁺ and Hg⁺ affected the enzyme activity considerably. The enzyme doesn't require calcium ion for its activity and/or stability (Table. 1).

TABLE 1
Effect of metal ions on protease activity

| Metal ions | Enzyme activity (U/mg precipitate) |
|---|---|
| None | 42.77 |
| CaCl₂ | 42.34 |
| CuSO₄ | 10.69 |
| ZnSO₄ | 12.40 |
| FeSO₄ | 37.64 |
| NaCl | 41.06 |
| MgSO₄ | 42.77 |
| MnSO₄ | 37.21 |
| HgCl₂ | 9.83 & 6.84 |

All metal ions at 5 mM concentration and HgCl₂ at 1 & 5 mM

### EXAMPLE-23

This example illustrates the effect of ingredients of detergents on activity of the protease isolated from Pseudomonas aeruginosa MCM B-327 when grown in soybean meal−tryptone (HiMedia) medium. The cell free supernatant was salt precipitated and used for this study in Tris-HCl buffer, pH 8.0 at 37° C. The enzyme and ingredients of detergent were incubated for 30 min prior to enzyme assay. The enzyme was stable in detergent ingredients while 20% inhibited by SDS. Surfactant like Tween 80 and Triton X100 had no inhibitory effect on protease activity (Table. 2).

TABLE 2
Effect of ingredients of detergents on protease activity

| Detergents ingredients (1%) | Enzyme activity (U/mg precipitate) |
|---|---|
| None | 42.77 |
| SDS | 34.21 |
| Sodium tripolyphosphate | 44.05 |
| Sodium tetra borate | 41.91 |
| Tween 80 | 43.62 |
| Triton X100 | 41.49 |

This example illustrates the effect of commercial detergents on activity of the protease isolated from Pseudomonas aeruginosa MCM B-327 when grown in soybean meal−tryptone (HiMedia) medium. The cell free supernatant was salt precipitated and used for this study in Tris-HCl buffer, pH 8.0 at 37° C. The enzyme and the commercial detergent (1:5 ratios) were incubated for 30 min prior to enzyme assay. The enzyme showed 69-92% activity in proprietary commercial detergents like Tide®, Ariel®, Nirma®, Rin Shakti® and Surf Excel® (Table. 3).

TABLE 3
Effect of commercial detergents on protease activity

| Commercial detergents (1%) | Enzyme activity (U/mg precipitate) |
|---|---|
| None | 15.49 |
| Tide ® | 10.69 |
| Ariel ® | 11.15 |
| Nirma ® | 11.46 |
| Rin Shakti ® | 13.16 |
| Surf excel ® | 14.25 |

### EXAMPLE-25

This example illustrates the effect of inhibitors on activity of the protease isolated from Pseudomonas aeruginosa MCM B-327 when grown in soybean meal−tryptone (HiMedia) medium. The cell free supernatant was salt precipitated and used for this study in Tris-HCl buffer, pH 8.0 at 37° C. The enzyme and the inhibitor were incubated for 30 min prior to enzyme assay. The enzyme was completely inhibited by DTT. The enzyme was 25-30% inhibited by PMSF and EDTA at 5 mM concentration (Table. 4).

TABLE 4
Effect of inhibitors on protease activity

| Inhibitors | Concentration | Enzyme activity (U/mg precipitate) |
|---|---|---|
| None | — | 42.77 |
| EDTA | 2 & 5 mM | 32.93 & 30.36 |
| PMSF | 1 & 5 mM | 40.20 & 31.65 |
| Iodoacetamide | 1 & 5 mM | 44.05 & 43.62 |
| DTT | 1 & 5 mM | 0 |
| Trypsin inhibitor | 100 μg | 37.21 |

(EDTA—Ethylenediamine tetra acetic acid; PMSF—Phenyl methyl sulphonyl fluoride, DTT—dithiothretol)

### EXAMPLE-26

This example illustrates the effect of organic solvents on activity of the protease isolated from Pseudomonas aeruginosa MCM B-327 when grown in soybean meal−tryptone (HiMedia) medium. The cell free supernatant was salt precipitated and used for this study in Tris-HCl buffer, pH 8.0 at 37° C. The enzyme showed more than 60% activity in n-hexane, isooctane and m-p-xylene at 50% v/v concentration after 30 min of incubation. The enzyme was 51% inhibited by toluene, 70-75% inhibited by methanol, acetone, benzene, 1-pentanone and 91% inhibited by ethanol (Table. 5).

TABLE 5
Effect of organic solvents on protease activity

| Organic solvents (50%) | Enzyme activity (U/mg precipitate) |
|---|---|
| None | 42.77 |
| n-hexane | 26.94 |
| Isooctane | 28.65 |
| m-p-xylene | 28.65 |
| Toluene | 20.95 |
| Methanol | 14.11 |
| Acetone | 13.68 |
| Benzene | 11.54 |
| 1-pentanone | 11.54 |
| Ethanol | 3.84 |

### EXAMPLE-27

This example illustrates the application of enzyme protease for dehairing of goatskins. Wet salted goatskin of 1.5 Kg was used for dehairing. The presoak was carried out with 300% water in a pit method. The soaked skin was handled twice in an hour. Enzyme, 1% (w/w of skin) was mixed with 10% water and applied on the flesh side of the skin. After 15-18 hrs, dehairing was carried out using blunt knife.

Visual assessment studies of enzyme dehaired pelt revealed the complete removal of hair. The pelt was white in color than the control in which dehairing was carried out using conventional chemical method. Physical testing results showed that the leather obtained using protease was comparable to that obtained by lime and sulphide. Histopathalogical studies of the dehaired pelt produced using protease revealed that the epidermis was totally removed with partly opened up of collagen bundles with empty follicles while conventional dehairing showed hair follicle and dense collagen bundles.

### EXAMPLE-28

This example illustrates the application of enzyme protease for dehairing of buffalo hide. Wet salted buffalo hide of approximately 5 Kg was used for dehairing. Samples from neck region of the animal having higher thickness of the hide were also included. The presoak was carried out with 300% water in a pit method. The soaked hide was periodically handled up to 24 hrs. The paste of 3% enzyme in 10% water was applied on grain side of the hide. After 18-21 hrs, dehairing was carried out using blunt knife.

Visual assessment studies of enzyme dehaired pelt revealed the complete removal of hair. The pelt was white than the control where dehairing was carried out using conventional chemical method. Physical testing results showed that the leather obtained using protease was comparable to those obtained by lime and sulphide. Histopathalogical studies of the dehaired pelt produced using protease revealed that the epidermis was totally removed with partly opened up of collagen bundles with empty follicles while conventional dehairing showed hair follicle and dense collagen bundles.

### Advantages

The main advantages of the present invention are the following.
a) The protease of the present invention is produced with cheap substrates to reduce the cost of production.
b) The protease of the present invention is active in wider pH range of 6-11 and temperature range of 25-65° C.
c) The protease of the present invention is found to be stable in presence of SDS, sodium tripolyphosphate, sodium tetraborate, tween 80, triton X100 which are ingredients of commercial detergents and surfactants. It is also active in proprietary commercial detergents like Tide®, Ariel®, Nirma®, Rin Shakti® and Surf Excel®.
d) The protease of the present invention is found to be calcium ion independent for its activity and/or stability.
e) The protease of the present invention is found to be effective for dehairing of animal skins as well as hides.
f) Enzymatically prepared wet blue and crust leather were of comparable quality with control.
g) The protease of the present invention can improve the leather quality, reduce the pollution load of the tannery effluent and avoid the health hazards to the tannery workers.
h) The present protease results in dehairing of animal hide from hair root yielding intact hair that can be used as a byproduct.
i) The protease of present invention is found to hydrolyze albumin hence could be used in soaking of an animal hide.

1. A novel protease obtained from a bacterial strain of Pseudomonas aeruginosa MCM B-327, deposited at MTCC, IMTECH, Chandigarh, India and designated as MTCC 5270, having the following characteristics:
(a) molecular weight in the range of 55 to 70 kDa;
(b) specificity against globular type of proteins;
(c) stability in pH range of 6.0 to 10.0 and temperature ranging from 25 to 45 degree C.;
(d) activity in pH range of 6.0 to 11.0 and temperature ranging from 25 to 65 degree C.;
(e) the said protease is calcium ion independent for its activity and/or stability;
(f) the said protease exhibits inhibition by metals Cu⁺², Zn⁺², H^{g+1}, and solvents;
(g) the said protease exhibits inertness to collagen.

2. A novel protease according to claim 1, wherein the substrates thereof are selected from the group consisting of casein, bovine serum albumin, gelatin, azocasein, azoalbumin, azocoll.

3. A novel protease according to claim 1, wherein the protease is inert to fibrous protein-collagen.

4. A novel protease according to claim 1, wherein the stability of the protease is independent of divalent metal ions such as Ca⁺² and Mg+2.

5. A novel protease according to claim 1, wherein it shows inhibition in the presence of metals such as Cu⁺², Zn⁺², Hg⁺¹.

6. A novel protease according to claim 1, wherein it shows inhibition in the presence of solvents such as methanol, benzene, 1-pentanone and ethanol.

7. A novel protease according to claim 1, wherein the said protease is useful in dehairing of animal skins and hides, as a detergent additive, in preparation of protein hydrolysate and recovery of silver from X-Ray and photographic films.

8. A process for the preparation of a novel protease according to claim 1, wherein the said process comprises:
c) growing Pseudomonas aeruginosa MCM B-327, deposited at MTCC, IMTECH, Chandigarh and designated as MTCC 5270 in a production medium containing essentially a protein source, an organic nitrogenous source, maintained at a pH in the range of 6.0 to 9.0, in submerged culture condition, at temperature ranging from 25 to 40 degree C., under shaking condition to obtain a culture broth;
d) harvesting the culture broth obtained from step (a) after a period in the range of 48 to 72 hrs followed by separation of the enzyme in liquid phase by salting out method to obtain the novel protease.

9. A process according to claim 8, wherein the bacterial strain used is isolated from vermicompost pit soil, Pune, Maharashtra, India.

10. A process according to claim 8, wherein the protein source used for the production medium is selected from agricultural products/ byproducts such as deoiled ground nut cake, saffola cake, rape seed cake, soybean cake, soya flour, soybean meal, bengal gram flour, wheat bran, either individually or in any combination.

11. A process according to claim 8, wherein the organic nitrogenous source used for the culture medium is selected from beef extract, yeast extract, peptone, tryptone and casein either individually or in any combination.

12. A process according to claim 8, wherein the method of separation of protease used is such as ammonium sulphate precipitation.

13. A process according to claim 8, wherein 1% each of soybean meal and tryptone are used as raw materials in the production medium for the preparation of the protease at a pH of 8.0, inoculum density of 10⁹ CFU/ml of P. aeruginosa MTCC 5270, at a temperature of 30 degree C. for a period of 72 hours under shake culture conditions.

14. A process according to claim 8, wherein the said protease is useful in dehairing of animal skins and hides, as a detergent additive, in preparation of protein hydrolysate and recovery of silver from X-Ray and photographic films.
